(19) United States
(12) Patent Application Publication
(10) Pub. No.: US 2022/0403006 A1
(43) Pub. Date: Dec. 22, 2022

(54) A Precision Medicine Method for Immunotherapy

(71) Applicant: Edifice Health, Inc., San Mateo, CA (US)

(72) Inventor: David Furman, San Mateo, CA (US)

(73) Assignee: Edifice Health, Inc., San Mateo, CA (US)

(21) Appl. No.: 17/775,186

(22) PCT Filed: Nov. 6, 2020
(86) PCT No.: PCT/US2020/059349

§ 371 Date: May 6, 2022

Related U.S. Application Data
(60) Provisional application No. 62/933,981, filed on Nov. 12, 2019.

(51) Int. Cl. C07K 16/00; A61K 39/12

(52) U.S. Cl. CPC C07K 16/00; A61K 39/12

(57) ABSTRACT

Cancer immunotherapy has achieved immense clinical success with long survival even in the most difficult to treat cancer. Yet this effect is only observed in a minority and there are no biomarkers of this response. The methods described herein reduce immunosenescence, improve cancer immunotherapy outcomes, vaccination outcomes, and/or treatment of infectious diseases using two independent measures of systemic chronic inflammation (the inflammatory age—iAge—and cytokine response score—CRS) to stratify patients into responders versus non-responders to cancer immunotherapy, vaccination, and/or anti-pathogen therapies. The iAge personalized immune proteome signature creates an individualized initial therapy to reduce iAge and to convert nonresponders patients into responders prior to treatment. Nonresponders can be converted to responders by treating the patients to reduce their iAge and improve their CRS.

### BACKGROUND

Over the past five years cancer immunotherapy treatments have witnessed a great deal of clinical success in multiple cancer types often with extended disease-free survival periods of >10 years. Examples of successful immunotherapies are immune checkpoint inhibitors, which have demonstrated unprecedented rates of durable responses in many difficult-to-treat cancers. However, regardless of the organ affected and cancer type, only a limited percentage of patients (˜20%) benefit from these approaches. Thus, there is a growing need to identify biomarkers that will improve the selection of patients who will respond to therapy.

Biomarkers are needed both before and during treatment to enable identification of patients likely to respond to immunotherapy treatment in order to reduce inappropriate drug use. Objective clinical responses are defined as a reduction in tumor size during the course of treatment. Multiple baseline factors associated with disease prognosis have been linked to response rates. For example, patients with small-sized tumors or low baseline levels of serum lactate dehydrogenase (LDH) are more likely to respond to antiPD-1 treatment. Circulating tumor DNA (ctDNA) that can be released by dead tumor cells and detected in the serum of some patients correlate strongly with tumor progression.

Response to anti-PD-1 treatment can partially be predicted by the expression of the ligand PD-L1 within the tumor microenvironment. Although PD-L1 expression is correlated with treatment efficacy in melanoma patients, it is not in patients with other cancers such as squamous cell carcinoma, non-small cell lung cancer and Merkel cell carcinoma.

The presence of neoantigens on tumor cells promotes immunogenicity against tumors and improves treatment efficacy. Thus, high genetic variation between tumor cells and host cells is one indicator of checkpoint inhibitor treatment efficacy. This is particularly true for anti-CTLA-4 treatment in melanoma patients and anti-PD-1 treatment in patients with colorectal cancer or non-small cell lung cancer with high mutation rates.

Other immunological factors associated with improved treatment responses prior to immunotherapy treatment include elevated eosinophil and lymphocyte counts; high numbers of CD8⁺ T cells infiltrating the tumor, and increased TGF-β levels in the serum from melanoma patients treated with anti-PD-1.

A number of post-treatment immune biomarkers have also been suggested to be associated with improved responses to cancer immunotherapy. For instance, patients who were more likely to respond to anti-CTLA-4 treatment had increased counts of inducible co-stimulatory molecule (ICOS)(+) T cells and lower neutrophil/lymphocyte ratios.

Described herein is a method for treating cancer utilizing patient stratification based on the intracellular or extracellular levels of chronic inflammation (iAge, cytokine response score CRS, and/or Jak-STAT response) of a subject followed by the design of individualized therapies aimed to improve the clinical and immunological responses to cancer immunotherapy.

### SUMMARY

The disclosure describes a method for treating subjects with immunotherapy (e.g., cancer patients), vaccines (e.g., subjects who will benefit from vaccination), and antipathogen therapeutics (e.g., antibiotics, antivirals, antifungals, etc.) whereby subjects can be stratified based on their inflammatory age levels; and can receive individualized interventions to reduce inflammatory age and improve clinical and immune responses to the therapeutic treatment (e.g., cancer immunotherapy, vaccination, anti-pathogen therapeutic).

An inflammatory age scoring system (iAge) can be used to classify subjects (e.g., cancer patients, vaccination subjects, subjects with an infectious disease) into those who have an immune system that can mount an effective response (e.g., mount an objective clinical response to immunotherapy, produce a protective response to a vaccine, or mount an immune response against a pathogen) versus those who will not. The inflammatory age scoring system can be used to guide initial therapy targeting inflammation to enable optimal objective responses in those patients who were classified as non-responders. The iAge can also be used to stratify subjects for different courses of vaccines or antipathogen therapy. A cytokine response score (CRS) can also be used to classify cancer patients into those who will mount an objective clinical response to immunotherapy versus those who will not.

Based on a subject's iAge, CRS, and/or Jak-STAT responses the subject can be classified as a responder or a nonresponder for the immunotherapy, vaccination, and/or anti-pathogen therapy. Patients who are classified as nonresponders can be treated to lower their iAge, increase their CRS, and/or increase their Jak-STAT response so that the subject moves into a responder category. Nonresponders can also be stratified to receive other anti-cancer therapies, other vaccines, or other anti-pathogen therapies. Classifications are made by comparing the subjects iAge, CRS, and/or Jak-STAT response to those of patients of similar chronological age. When a subject's iAge, CRS, and/or Jak-STAT response places them at a younger iAge for their age cohort, or a more responsive CRS and/or Jak-STAT score the subject can be a responder for immunotherapy, vaccination, or anti-pathogen therapy. Subjects with older iAge for their age cohort, and/or lower scores for CRS and/or Jak-STAT can be treated to lower their iAge and/or increase their CRS and/or Jak-STAT score so that they move into a responder group for immunotherapy, vaccination or anti-pathogen therapy.

### BRIEF DESCRIPTION OF THE DRAWINGS

FIGS. 1A, 1B and 1C show graphs of iAge, naïve CD8(+) T-cells, and Jak STAT signaling responses.

FIG. 2 shows the stratification of cancer patients by iAge and CRS into responders and nonresponders.

FIG. 3 shows the stratification of cancer patients using iAge.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, “activation” is defined to be a physiological condition upon exposure to a substance, allergen, drug, protein, chemical, or other stimulus, or upon removal of a substance, allergen, drug, protein, chemical or other stimulus.

As used herein, an “antibody” is defined to be a protein functionally defined as a ligand-binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

As used herein, an “antibody fragment” is defined to be at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either V_{L} or V_{H}), camelid VHH domains, and multispecific antibodies formed from antibody fragments. The term “scFv” is defined to be a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

As used herein, an “antigen” is defined to be a molecule that provokes an immune response. This immune response may involve either antibody production, or the activation of specific immunologically-competent cells, or both. The skilled artisan will understand that any macromolecule, including, but not limited to, virtually all proteins or peptides, including glycosylated polypeptides, phosphorylated polypeptides, and other post-translation modified polypeptides including polypeptides modified with lipids, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or can be a macromolecule besides a polypeptide. Such a biological sample can include, but is not limited to a tissue sample, a tumor sample, a cell or a fluid with other biological components.

As used herein, the terms “Chimeric Antigen Receptor” and the term “CAR” are used interchangeably. As used herein, a “CAR” is defined to be a fusion protein comprising antigen recognition moieties and cell-activation elements.

As used herein, a “CAR T-cell” or “CAR T-lymphocyte” are used interchangeably, and are defined to be a T-cell containing the capability of producing CAR polypeptide, regardless of actual expression level. For example a cell that is capable of expressing a CAR is a T-cell containing nucleic acid sequences for the expression of the CAR in the cell.

As used herein, an “effective amount” or “therapeutically effective amount” are used interchangeably, and defined to be an amount of a compound, formulation, material, or composition, as described herein effective to achieve a particular biological result.

As used herein, an “epitope” is defined to be the portion of an antigen capable of eliciting an immune response, or the portion of an antigen that binds to an antibody. Epitopes can be a protein sequence or subsequence that is recognized by an antibody.

As used herein, an “expression vector” and an “expression construct” are used interchangeably, and are both defined to be a plasmid, virus, or other nucleic acid designed for protein expression in a cell. The vector or construct is used to introduce a gene into a host cell whereby the vector will interact with polymerases in the cell to express the protein encoded in the vector/construct. The expression vector and/or expression construct may exist in the cell extrachromosomally or integrated into the chromosome. When integrated into the chromosome the nucleic acids comprising the expression vector or expression construct will be an expression vector or expression construct.

As used herein, “heterologous” is defined to mean the nucleic acid and/or polypeptide are not homologous to the host cell. For example, a construct is heterologous to a host cell if it contains some homologous sequences arranged in a manner not found in the host cell and/or the construct contains some heterologous sequences not found in the host cell.

As used herein, the term “impaired immune function” is defined to be any reduction in immune function in an individual, as compared to a fully healthy individual. Individuals with an impaired immune function are readily identifiable by substantially increased abundance of CD8+CD28− cells or more broadly by reduced cytokine responses, increased baseline phosphoprotein levels and other co-occurring measure.

As used herein, the term “inflammasome” is defined as cytosolic multiprotein complexes that are composed of an inflammasome-initiating sensor, apoptosis-associated speck-like protein containing a CARD (Caspase Activation and Recruitment Domain) acts as an adaptor protein and the protease-caspase-1. Inflammasome-initiating sensors include members of the NLRs the pyrin and HIN domain-containing (also known as PYHIN, Aim 2-like receptors, or ALRs; e.g., Aim2), or the TRIM (e.g., pyrin) family. Complex assembly leads to caspase-1-dependent cleavage of cytokines pro-interleukin 1β (pro-IL-1β) and pro-IL-18 into secreted mature forms. In addition, inflammasomes initiate pyroptotic cell death.

As used herein, a “single chain antibody” (scFv) is defined as an immunoglobulin molecule with function in antigen-binding activities. An antibody in scFv (single chain fragment variable) format consists of variable regions of heavy (V_{H}) and light (V_{L}) chains, which are joined together by a flexible peptide linker.

### Immunological Age

The Jak/STAT signaling pathway is critical for meeting the multiple challenges encountered by the immune system, from fighting infections to maintaining immune tolerance. Clearly STATs are also involved in the development and function of the immune system in humans and play a key role in maintaining immune surveillance of cancer (Nature. 2007; 450(7171):903-7; Nat Rev Cancer (2009) 9:798-809).

The Jak-STAT pathway can be profoundly altered with aging and this is one major cause of immune dysfunction in older adults. A cytokine response score (CRS) can be used to predict immune decline and reduction in immune surveillance of cancer.

An inflammatory age scoring system (iAge) can also be used to predict ageassociated multimorbidity and mortality. iAge can be extremely sensitive as a biomarker of cardiovascular health since elevated levels predict left ventricular remodeling and arterial stiffness even in very healthy older subject with no clinical or laboratory cardiovascular risk factors. iAge can also identify subclinical immunodeficient young patients (10% of subjects 16-35 years old) who cannot mount responses to any strain of the influenza vaccine in any of the years studied (up to 6 years follow-up). These subjects are characterized by having an older-like immunological phenotype with regards to their immune cell composition, ex vivo responses to multiple acute stimuli, and expression of gene modules associated with advanced age.

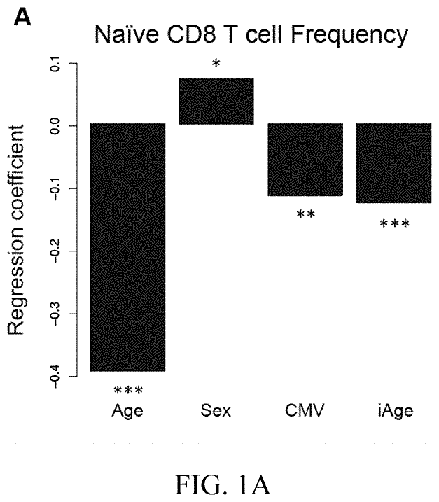

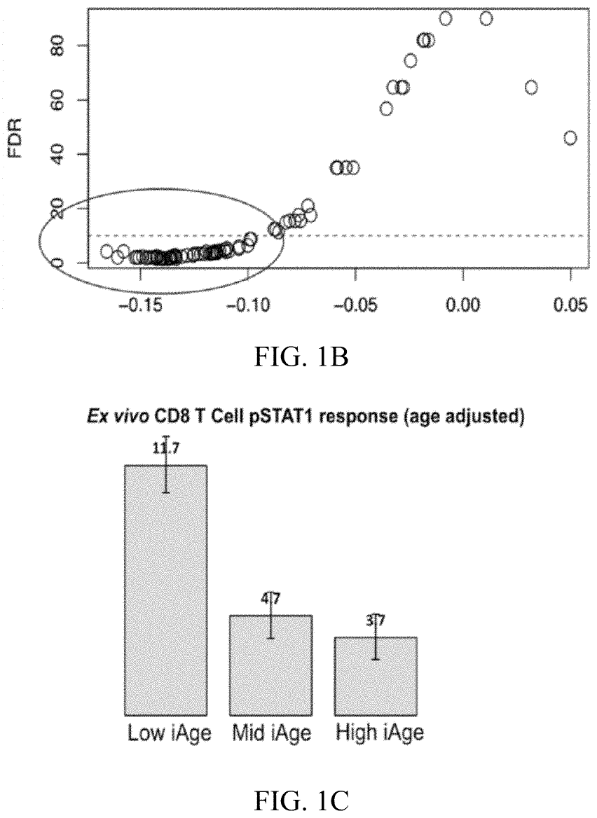

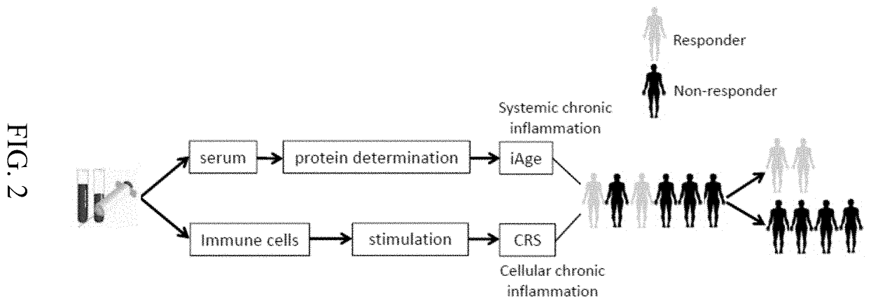

Since the cytokine response score CRS and iAge are independent measures of inflammation, diminished Jak-STAT signaling pathway in T cells, and low naïve CD8(+) T cell counts (FIG. 1A-C) these measures can be used to stratify cancer patients with respect to their clinical responses to immunotherapy. The methods described herein use blood inflammatory markers CRS and iAge to stratify cancer patients into responder and nonresponders groups for immunotherapy. The nonresponders can be treated to reduce their iAge and/or increase their CRS (and/or Jak-STAT score) so that the nonresponders obtain iAge and/or CRS (and/or Jak-STAT score) that places them into a responder group.

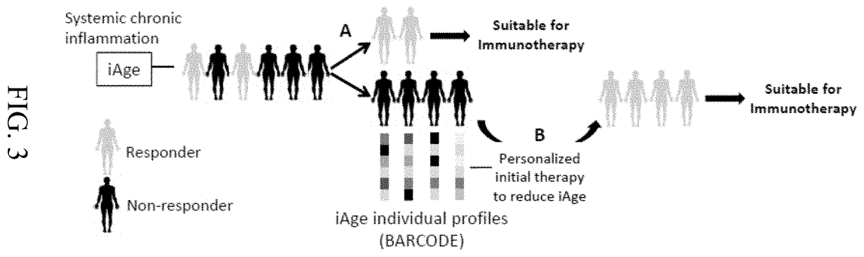

The procedure involves the extraction of peripheral blood samples by venipuncture, or by any appropriate method, from candidate cancer patients prior to infusion with immunotherapy treatment (FIG. 2). Immunotherapy treatment may comprise the use of certain molecules including antibodies, small molecules, etc. against inhibitory immune receptors. Blood serum is separated from blood cells by centrifugation of clogged blood, or by any other appropriate method (FIG. 2).

Construction of iAge: For serum protein determination, the resulting sera can be mixed with antibody-linked magnetic beads on 96-well filter-bottom plates and can be incubated at room temperature for 2 h followed by overnight incubation at 4° C. Room temperature incubation steps can be performed on an orbital shaker at 500-600 rpm. Plates can be vacuum filtered and washed twice with wash buffer, then incubated with biotinylated detection antibody for 2 h at room temperature. Samples can be then filtered and washed twice as above and re-suspended in streptavidin-PE. After incubation for 40 minutes at room temperature, two additional vacuum washes can be performed, and the samples can be re-suspended in Reading Buffer. Each sample can be measured in duplicate or triplicate. Plates can be read using a Luminex 200 instrument with a lower bound of 100 beads per sample per cytokine and mean fluorescence intensity (MFI) is recorded.

To derive inflammatory age (iAge) (FIG. 2), the mean fluorescence intensity can be normalized and used for multiple regression analysis, which is computed using the following regression coefficients: MIG: 0.6357, TRAIL: −0.3760, IFNG: −0.3235, EOTAXIN: 0.2912, GROA: −0.2723, IL2: −0.2063, TGFA: −0.1978, PAI1: −0.1587, LIF: −0.1587, LEPTIN: 0.1549, MIP1A: 0.1547, IL1B: 0.1471. The MFI can be multiplied by the regression coefficient for the protein, and these numbers can be all added together to give the iAge of the subject. Table 1 below lists the ranges of iAge within chronological age decades.

TABLE 1
iAge Ranges

| Chronological Age (years) | iAge Range |
|---|---|
| 10-19 | 18.1-58.3 |
| 20-29 | 18.5-78.9 |
| 30-39 | 16.9-70.2 |
| 40-49 | 21.5-74.1 |
| 50-59 | 23.1-74.4 |
| 60-69 | 28.1-76.6 |
| 70-79 | 35.6-77.6 |
| 80-96 | 37.9-78.5 |

Those markers with positive regression coefficients increased in serum concentration with age (MIG, EOTAXIN, LEPTIN, MIP1A, and IL1B) and those with negative regression coefficients decreased in serum concentration with age (TRAIL, IFNG, GROA, IL2, TGFA, PAD, and LIF).

MIG (monokine induced by gamma interferon) is a small cytokine belonging to the CXC chemokine family. MIG is one of the chemokines which plays a role to induce chemotaxis, promote differentiation and multiplication of leukocytes, and cause tissue extravasation. MIG regulates immune cell migration, differentiation, and activation. Tumor-infiltrating lymphocytes are a key for clinical outcomes and prediction of the response to checkpoint inhibitors. In vivo studies suggest the axis plays a tumorigenic role by increasing tumor proliferation and metastasis. MIG predominantly mediates lymphocytic infiltration to the focal sites and suppresses tumor growth.

TRAIL (TNF-related apoptosis-inducing ligand) is a cytokine that is produced and secreted by most normal tissue cells. It is thought to cause apoptosis primarily in tumor cells by binding to certain death receptors. TRAIL has also been designated CD253 (cluster of differentiation 253) and TNFSFlO (tumor necrosis factor (ligand) superfamily, member 10). TRAIL is described in Wiley et al Immunity 1005 3: 673-82 as well as Pitti J. Biol. Chem. 1996 271: 12687-90.

INFG (otherwise known as interferon gamma, IFNy or type II interferon) is a dimerized soluble cytokine that is the only member of the type II class of interferons. IFNG is critical for innate and adaptive immunity against viral, some bacterial and protozoan infections. INFG is an important activator of macrophages and inducer of Class II major histocompatibility complex (MHC) molecule expression. INFG is described In Schoenborn et al Adv. Immunol. 2007 96: 41-101 as well as Gray Nature. 1982 298: 859-63.

Eotaxin (also known as C-C motif chemokine I I or eosinophil chemotactic protein) is a small cytokine belonging to the CC chemokine family. Eotaxin selectively recruits eosinophils by inducing their chemotaxis, and therefore, is implicated in allergic responses. The effects of eotaxin is mediated by its binding to a G-protein-linked receptor known as a chemokine receptor. Chemokine receptors for which CCLI I is a ligand include CCR2, CCR3 and CCR5. Eotaxin is described in Kitaura et al The Journal of Biological Chemistry I 996 271: 7725-30 and Jose et al The Journal of Experimental Medicine 1994 I 79: 881-7.

GROA (also known as CXCLI, the GROI oncogene, GROa, KC, neutrophilactivating protein 3 (NAP-3) and melanoma growth stimulating activity, alpha (MSGA-a)) is secreted by human melanoma cells, has mitogenic properties and is implicated in melanoma pathogenesis. GROA is expressed by macrophages, neutrophils and epithelial cells, and has neutrophil chemoattractant activity. This chemokine elicits its effects by signaling through the chemokine receptor CXCR2. GROA is described in Haskill et al Proc. Natl. Acad. Sci. U.S.A. 190 87 (19): 7732-6.

IL-2 is one of the key cytokines with pleiotropic effects on the immune system. It is a 15.5-16 kDa protein that regulates the activities of white blood cells (leukocytes, often lymphocytes) that are responsible for immunity. The major sources of IL-2 are activated CD4+ T cells, activated CD8+ T cells, NK cells, dendritic cells and macrophages. IL-2 is an important factor for the maintenance of CD4+ regulatory T cells and plays a critical role in the differentiation of CD4+ T cells into a variety of subsets. It can promote CD8+ T-cell and NK cell cytotoxicity activity, and modulate T-cell differentiation programs in response to antigen, promoting naive CD4+ T-cell differentiation into T helper-1 (Th1) and T helper-2 (Th2) cells while inhibiting T helper17 (Th17) differentiation.

TGFA (transforming growth factor alpha) is a polypeptide of 5.7 kDa that is partially homologous to EGF. TGFA is a growth factor that is a ligand for the epidermal growth factor receptor, which activates a signaling pathway for cell proliferation, differentiation and development. TGFA also is a potent stimulator of cell migration. TGFA can be produced in macrophages, brain cells, and keratinocytes. TGFA can induce epithelial development. TGFA can also upregulate TLR expression and function augmenting host cell defense mechanisms at epithelial surfaces. TGFA may act as either a transmembrane-bound ligand or a soluble ligand. TGFA has been associated with many types of cancers, and it may also be involved in some cases of cleft lip/palate. Alternatively spliced transcript variants encoding different isoforms have been found for this gene.

PALL (plasminogen activator inhibitor-1) is a member of the serine proteinase inhibitor (serpin) superfamily. PAI1 is the principal inhibitor of tissue plasminogen activator (tPA) and urokinase (uPA), and hence is an inhibitor of fibrinolysis. PALL is also a regulator of cell migration. PAI1 can play a role in a number of age-related, conditions including, for example, inflammation, atherosclerosis, insulin resistance, obesity, comorbidities, and Werner syndrome. PAI1 can play a host protective role during the acute phase of infection by regulating interferon gamma release. IFNG regulates PAI-1 expression, which suggests an intricate interplay between PAI-1 and IFNG. PAI1 can also activate macrophages through Toll-like receptor 4 (TLR4) and can promote migration of pro-cancer M2 macrophages into tumors.

LIF (leukemia inhibitory factor) is interleukin 6 class cytokine with pleiotropic effects impacting several different systems. LIF is involved in the induction of hematopoietic differentiation in normal and myeloid leukemia cells, induction of neuronal cell differentiation, regulator of mesenchymal to epithelial conversion during kidney development, and may also have a role in immune tolerance at the maternal-fetal interface. LIF can introduce and/or maintain epigenetic plasticity within a cells genome, i.e., LIF is permissive for epigenetic flexibility. This flexibility is greatest in stem cells where LIF functions in concert with Nanog, to choreograph pluripotency and selfrenewal in embryonic stem cells. LIF also plays a role in T-cell development into Tregs versus TH17 cells. Stem cells including mesenchymal stem cells are maintained in a pluripotent state by LIF and are immune privileged where LIF, but not IL-6, promotes stem cell expansion. Alternatively spliced transcript variants encoding multiple isoforms have been observed for this gene.

LEPTIN is secreted by white adipocytes into the circulation and plays a major role in the regulation of energy homeostasis. LEPTIN binds to the leptin receptor in the brain, which activates downstream signaling pathways that inhibit feeding and promote energy expenditure. LEPTIN also has several endocrine functions, and is involved in the regulation of immune and inflammatory responses, hematopoiesis, angiogenesis, reproduction, bone formation and wound healing. LEPTIN can directly link nutritional status and pro-inflammatory T helper 1 immune responses, and a decrease of LEPTIN plasma concentration during food deprivation can lead to an impaired immune function. LEPTIN is associated with the pathogenesis of chronic inflammation, and elevated circulating LEPTIN levels in obesity appear to contribute to low-grade inflammation which makes obese individuals more susceptible to increased risk of developing cardiovascular diseases, type II diabetes, and degenerative disease including autoimmunity and cancer. Reduced levels of LEPTIN such as those found in malnourished individuals have been linked to increased risk of infection and reduced cellmediated immune responses. Mutations in this gene and its regulatory regions cause severe obesity and morbid obesity with hypogonadism in human patients. A mutation in this gene has also been linked to type 2 diabetes mellitus development.

MIP1A (macrophage inflammatory protein) is a member of the CC or beta chemokine subfamily. MIP1A regulates leukocyte activation and trafficking. MIP1A acts as a chemoattractant to a variety of cells including monocytes, T cells, B cells and eosinophils. MIP1A plays a role in inflammatory responses through binding to the receptors CCR1, CCR4 and CCR5.

IL-1B (Interleukin-1 beta) is a member of the interleukin 1 cytokine family. IL1B is an important mediator of the inflammatory response, and is involved in a variety of cellular activities, including cell proliferation, differentiation, and apoptosis. LI-1B is produced by activated macrophages as a proprotein, which is proteolytically processed to its active form by caspase 1 (CASP1/ICE).

Construction of CRS: Separation of immune cells may comprise the use of differential centrifugation of blood by density gradient (FIG. 2). The resulting cell pellet can be suspended in warm media, wash twice and resuspended at 0.5×10\hat{ }6 viable cells/mL. 200 uL of cell sample can be plated per well in 96-well deep-well plates. After resting for 1 hour at 37° C., cells can be stimulated by adding 50 ul of cytokine (IFNa, IFNg, IL-6, IL-7, IL-10, IL-2, or IL-21) (FIG. 2) and incubated at 37° C. for 15 minutes. The cells can be fixed with paraformaldehyde, permeabilized with methanol, and kept at 80 C overnight. Each well can then be bar-coded using a combination of Pacific Orange and Alexa-750 dyes (Invitrogen, Carlsbad, Calif.) and pooled in tubes. The cells can be washed with FACS buffer (PBS supplemented with 2% FBS and 0.1% soium azide), and stained with the following antibodies (all from BD Biosciences, San Jose, Calif.): CD3 Pacific Blue, CD4 PerCP-Cy5.5, CD20 PerCp-Cy5.5, CD33 PE-Cy7, CD45RA Qdot 605, pSTAT-1 AlexaFluor488, pSTAT-3 AlexaFluor647, pSTAT-5 PE. The samples can be washed and resuspended in FACS buffer. 100,000 cells per stimulation condition are collected using DIVA 6.0 software on an LSRII flow cytometer (BD Biosciences). Data analysis can be performed using FlowJo v9.3 by gating on live cells based on forward versus side scatter profiles, then on singlets using forward scatter area versus height, followed by cell subset-specific gating.

Fold-change difference due to stimulation can be computed as the ratio of the cell, cytokine stimulation, phosphoprotein measure to the raw, un-normalized, cellphosphoprotein matching baseline that was measured on the same plate. The data can be normalized by scaling individual's by the average of the assay on the day in which they were measured.

To construct the Cytokine Response Score (CRS) (FIG. 2) 15 reproducible ageassociated normalized cytokine responses can be expressed as fold increases over baseline (unstimulated) and the fold increases for the following can be summed: CD8+ cells, stimulate with IFNa and measure pSTAT1, 3 and 5; CD8+ cells, stimulate with IL6 and measure pSTAT1, 3 and 5, CD8+ cells, stimulate with IFNg and measure pSTAT1, CD8+ cells, stimulate with IL21 and measure pSTAT1; CD4+ cells, stimulate with IFNa and measure pSTAT5, CD4+ cells, stimulate with IL6 and measure pSTAT5, CD20+ cells, stimulate with IFNa and measure pSTAT1, Monocytes stimulate with IL10 and measure pSTAT3, Monocytes stimulate with IFNg and measure pSTAT3, Monocytes stimulate with IFNa and measure pSTAT3, and Monocytes stimulate with IL6 and measure pSTAT3.

IFNA (Interferon alpha) is a member of the type I interferon class. And has thirteen (13) variants in humans. IFNA is secreted by hematopoietic cells, predominately plasmacytoid dendritic cells. IFNA can have either protective or deleterious roles. IFNA can be induced by ssRNA, dsRNA, and cytosolic DNA from viruses or bacteria. IFNA can induce caspase-11 expression, which contributes to activation of non-canonical inflammasome. Use of recombinant IFNA has been shown to be effective in reducing the symptoms and duration of the common cold.

INFG (Interferon gamma) is a member of the type II interferon class. The encoded protein is secreted by cells of both the innate and adaptive immune systems. The active protein is a homodimer that binds to the interferon gamma receptor which triggers a cellular response to viral and microbial infections. Mutations in this gene are associated with an increased susceptibility to viral, bacterial and parasitic infections and to several autoimmune diseases.

IL6 is a cytokine with pleiotropic effects on inflammation, immune response, and hematopoiesis. IL6 is promptly and transiently produced in response to infections and tissue injuries, contributes to host defense through the stimulation of acute phase responses, hematopoiesis, and immune reactions. IL6 functions in inflammation and the maturation of B cells. In addition, IL6 has been shown to be an endogenous pyrogen capable of inducing fever in people with autoimmune diseases or infections. IL6 is primarily produced at sites of acute and chronic inflammation, where it is secreted into the serum and induces a transcriptional inflammatory response through interleukin 6 receptor, alpha. IL6 is implicated in a wide variety of inflammation-associated disease states, including susceptibility to diabetes mellitus and systemic juvenile rheumatoid arthritis. Dysregulated, continual synthesis of IL-6 plays a pathological effect on chronic inflammation and autoimmunity. Alternative splicing results in multiple transcript variants.

IL10 is a cytokine with pleiotropic effects in immunoregulation and inflammation. IL-10 is an anti-inflammatory cytokine and during infection it inhibits the activity of Th1 cells, NK cells, and macrophages, all of which are required for optimal pathogen clearance but also contribute to tissue damage. IL10 can directly regulate innate and adaptive Th1 and Th2 responses by limiting T cell activation and differentiation in the lymph nodes as well as suppressing proinflammatory responses in tissues. It also enhances B cell survival, proliferation, and antibody production. This cytokine can block NF-kappa B activity, and is involved in the regulation of the JAKSTAT signaling pathway. Knockout studies in mice suggested the function of this cytokine as an essential immunoregulator in the intestinal tract.

IL21 is a member of the common-gamma chain family of cytokines with immunoregulatory activity. IL21 plays a role in both the innate and adaptive immune responses by inducing the differentiation, proliferation and activity of multiple target cells including macrophages, natural killer cells, B cells, cytotoxic T cells, and epithelial cells. IL21 is important to anti-tumor and antiviral responses and also exerts major effects on inflammatory responses that promote the development of autoimmune diseases and inflammatory disorders.

pSTAT1 (phosphorylated signal transducer and activator of transcription 1) mediates cellular responses to interferons (IFNs), cytokine KITLG/SCF and other cytokines and other growth factors. Following type I IFN (IFN-alpha and IFN-beta) binding to cell surface receptors, signaling via protein kinases leads to activation of Jak kinases (TYK2 and JAK1) and to tyrosine phosphorylation of STAT1 and STAT2. The phosphorylated STATs dimerize and associate with ISGF3G/IRF-9 to form a complex termed ISGF3 transcription factor, that enters the nucleus (PubMed:28753426). ISGF3 binds to the IFN stimulated response element (ISRE) to activate the transcription of IFNstimulated genes (ISG), which drive the cell in an antiviral state. In response to type II IFN (IFN-gamma), STAT1 is tyrosine- and serine-phosphorylated (PubMed:26479788). It then forms a homodimer termed IFN-gamma-activated factor (GAF), migrates into the nucleus and binds to the IFN gamma activated sequence (GAS) to drive the expression of the target genes, inducing a cellular antiviral state.

pSTAT 3 (phosphorylated signal transducer and activator of transcription 3) mediates cellular responses to interleukins, KITLG/SCF, LEP and other growth factors. Once activated, recruits coactivators, such as NCOA1 or MED1, to the promoter region of the target gene. Binds to the interleukin-6 (IL-6)-responsive elements identified in the promoters of various acute-phase protein genes. Activated by IL31 through IL31RA. Acts as a regulator of inflammatory response by regulating differentiation of naive CD4+ T-cells into T-helper Th17 or regulatory T-cells (Treg): deacetylation and oxidation of lysine residues by LOXL3, disrupts STAT3 dimerization and inhibits its transcription activity.

pSTAT 5 (phosphorylated signal transducer and activator of transcription 5) is activated by Janus-activated kinases (JAK) downstream of cytokine receptors. STAT5 proteins are activated by a wide variety of hematopoietic and nonhematopoietic cytokines and growth factors, all of which use the JAK-STAT signaling pathway as their main mode of signal transduction. STAT5 proteins critically regulate vital cellular functions such as proliferation, differentiation, and survival. STAT5 plays an important role in the maintenance of normal immune function and homeostasis, both of which are regulated by specific members of IL-2 family of cytokines, which share a common gamma chain (γ(c)) in their receptor complex. STAT5 critically mediates the biological actions of members of the γ(c) family of cytokines in the immune system. Essentially, STAT5 plays a critical role in the function and development of Tregs, and consistently activated STAT5 is associated with a suppression in antitumor immunity and an increase in proliferation, invasion, and survival of tumor cells.

### Immunosenescence

The immune system undergoes marked shifts in composition and function with aging, a pattern of changes that are together termed “immunosenescence”. Immunosenescence impacts both the innate and adaptive arms of the immune system and major features of immunosenescence include alteration in immune cell subset frequencies, defective antigen presentation, reduced cytotoxic function, and restricted T cell repertoire (Pawelec G, Larbi A. (2008), Immunity and ageing in man: Annual Review 2006/2007. Exper Gerontol 43:34-38; Weiskopf D, Weinberger B, GrubeckLoebenstein B. (2009), The aging of the immune system. Transpl Int. 22:1041-1050, both of which are incorporated by reference in their entirety for all purposes).

Other changes occurring during immunosenescence include reduction in cytokine signaling responses, increased baseline levels of phospho-STAT, and elevation in memory cell populations (Shen-Orr et al. (2016). Defective Signaling in the JAK-STAT Pathway Tracks with Chronic Inflammation and Cardiovascular Risk in Aging Humans, Cell Systems. 3(4):374-384.E4, which is incorporated by reference in its entirety for all purposes). Of notable clinical importance, immunosenescence also results in defects in antibody responses, with many older individuals failing to generate protective antibody titers following vaccination (Furman D et al. (2013). Apoptosis and other immune biomarkers predict influenza vaccine responsiveness. Molecular Systems Biology 9:659, which is incorporated by reference in its entirety for all purposes).

Immunosenescence impacts both the host's capacity to respond to infections and the development of long-term immune memory, especially by vaccination. Immunosenescence is associated with the accumulation of memory and effector cells as a result of repeated infections and by continuous exposure to antigens (inhalant allergens, food, etc.). This chronic inflammation characterizes immunosenescence and can have a significant impact on survival and fragility. Immunosenescence can also be associated with remodeling of the immune system caused by oxidative stress.

Immunosenescence can occur from an imbalance between inflammatory and antiinflammatory mechanisms producing chronic inflammation. This chronic inflammation can be due to chronic antigen stimulation occurring over the course of life and to the oxidative stress that involves the production of oxygen free radicals and toxic products. These factors are able to modify the potential of apoptotic lymphocytes, and this remodeling of the lymphocyte compartment and the chronic expression of proinflammatory cytokines are implicated in the processes of longevity and diseases related to immunosenescence.

Canonical acute inflammation proteins (C-reactive protein, Interleukin-6, etc.) have been associated with immunosenescence in previous studies, but the relationship with systemic chronic inflammation has not yet been established. Using a well-known marker for immunosenescence (the frequency of naïve CD8 (+) T cells) contribution of iAge to immunosenescence was estimated after controlling for Age, CMV, and sex by a multiple regression model. Age was the strongest contributor to changes in naïve CD8 (+) T cells followed by iAge, CMV (negative contributors) and sex (frequency of total CD8 (+) T cells in females was 24% vs. 30% in males). iAge was significantly correlated with the frequency of naïve CD8 (+) T cells to a similar extent to CMV positivity. Chronological age was the strongest contributor (P<10⁻¹⁵), followed by iAge (P<10⁻⁵), CMV (P<10⁻³) and gender (P=0.012) (A).

The effect of chronic inflammation on the immune response was also measured using a functional immune assay (phospho-flow) in which cells are stimulated ex vivo and the phosphorylation of various intracellular proteins is measured by using antibodies against phosphorylated forms of these proteins. In particular, the responses to four independent stimuli (Interferon-alpha, Interleukin-6, Interleukin-10 and Interleukin-21) were measured in a total of 818 individuals and the fold-increase in phospho-STAT1, -STAT3 and -STAT5 in B cells, total CD4 (+) T cells (and the CD45RA(+) and CD45RA(−) subsets), total CD8 (+) T cells (and the CD45RA(+) and CD45RA(−) subsets), and in monocytes were determined. Multiple regression analysis controlling age, CMV and sex, surprisingly showed there was a general decrease of the B cell and T cell responses to stimuli and an overall potentiation of the monocyte responses associated with increasing iAge (combined P<10⁻⁵).

These results demonstrate that iAge is an important immune predictor of immune function decline (immunosenescence) and can be used as a ‘metric’ for immunological health.

Immunosenescence is associated with lowered ability of the immune system to kill cancer cells, protect against infections from pathogenic organisms, and produce efficacious response to vaccines. Treating a subject to lower their iAge can reduce the immunosenescence in the subject and improve the ability of the subject's immune system to kill cancer cells, protect against infections from pathogenic organisms, and produce efficacious responses to vaccines. Agents and methods for lowering iAge and thereby reducing immunosenescence are described below.

### Immunotherapies

In recent years, there has been a sharp rise in the development and implementation of cancer immunotherapies against cancer. The approval of anticytotoxic T-lymphocyte-associated protein 4 (CTLA-4) and anti-programmed cell death protein 1 (PD-1) antibodies has resulted in significant improvements in disease outcomes for a variety of cancers. Unlike chemo- and radiotherapy, which aim to interfere with tumor cell growth and survival, immunotherapies indirectly target the tumor by boosting the anti-tumor immune responses of the patient. Despite the huge success of these therapies in many forms of cancer, the success rates are generally low and biomarkers to define objective clinical responses are still lacking.

The biological foundation of current cancer immunotherapies is the concept of cancer immune surveillance, which proposes that the immune system eliminates tumor cells because these possess new antigens and trigger an immune reaction with regression of the tumor and no clinical signs of its existence. In a seminal study to test this theory, it was shown that chemically-induced sarcomas grew faster and more aggressively in immune-incompetent mice than in wild type animals because the former lack lymphocytes (have a engineered mutation of the recombination-activating (RAG) gene) or their inability to respond to IFNγ, either because of the loss of the IFNγ receptor gene, or the STAT-1 gene. Immune surveillance was shown by using mice with a double mutation RAG2^{-/-}/STAT1^{-/-} which spontaneously developed tumors. These tumors resemble some of the major malignancies of humans, such as breast, lung, or colon. Cancer immunoediting was shown by transplanting tumors between mice. When a tumor was transplanted from an immune incompetent mouse to a immune competent mouse, 40% of the tumors were rejected. Whereas no rejection occured when transplants were performed using tumors from syngeneic immune-competent mice. This clearly demonstrated that immunoediting had occurred in immune-competent animals, even if they were incapable of rejecting their own tumor, enabling their escape from immunesurveillance. After decades of follow-up work a novel theory of tumor immunity was introduced. The theory proposed three steps: 1) elimination of tumors at early stages (immune surveillance hypothesis), 2) equilibrium which refers to the state in which the immune system controls the tumor, and 3) escape when tumor cells are immune-edited and grow without immune control. This three E's theory is still the theory accepted worldwide as the basis to understand the interaction of cancer cells with the immune system. The theory also paved the way for the exploding field of cancer immunotherapy.

Immunotherapy for cancer boosts the body's natural defenses to fight cancer. It uses substances made by the body or in a laboratory to improve or restore immune system function. Cancer immunotherapies include, for example, monoclonal antibodies, immune checkpoint inhibitors, cancer vaccines, immune cells modified with, for example, chimeric antigen receptors, and other nonspecific immunotherapies that boost the immune system function or action by, for example, specifically targeting cancer cells, overcoming inhibition of the immune system (e.g., by myeloid suppressor cells), etc.

Monoclonal antibodies for treating cancer include, for example, anti-CD20 antibody (e.g., Bexxar®, Zevalin®, Rituxan®, Gazyvaro®, Arzerra®), anti-Her2 antibody (e.g., Herceptin®, Kadcyla®, Perjeta®), anti-CD30 antibody (e.g., Adcetris®), anti-CD19 and anti-CD3 bispecific antibody (e.g., Blincyto®), anti-VegF antibody (e.g., Avastin®, Cyramza®), anti-EGFR antibody (e.g., Erbitux®, Portrazza®, Vectibix®), anti-PDGFR-α antibody (e.g., Lartruvo®), anti-CD38 antibody (e.g., Darzalex®), antiSLAMF7 antibody (e.g., Empliciti®), anti-GD2 antibody (e.g., Unituxin®), anti-CD19 antibody (e.g., Blincyto®), anti-RANKL antibody (e.g., Xgeva®, Prolia®), anti-EpCAM and anti-CD3 antibody (e.g., Removab®), anti-EpCAM antibody (e.g., Proxinium®), anti-CD52 antibody (e.g., Campath®), and anti-CD33 antibody (e.g., Mylotarg®).

Checkpoint inhibitors for treating cancer include, for example, Nivolumab (Opdivo), Pembrolizumab (Keytruda), Atezolizumab (Tecentriq), Ipilimumab (Yervoy), Durvalumab (Imfinzi®), Avelumab (Bavencio®), Lirilumab, and BMS-986016 (Relatlimab). Nivolumab, Atezolizumab, Pembrolizumab, Durvalumab, and Avelumab act at the checkpoint protein PD-1/PD-L1 and inhibit apoptosis of anti-tumor immune cells. Ipilimumab acts at CTLA4 and prevents CTLA4 from downregulating activated Tcells in the tumor. Lirilumab acts at KIR and facilitates activation of Natural Killer cells. BMS-986016 acts at LAG3 and activates antigen-specific T-lymphocytes and enhances cytotoxic T cell-mediated lysis of tumor cells.

Chimeric Antigen Receptors for treating cancer include, for example, an antiCD19 CAR in T-cells (e.g., Kymriah® and Yescarta®). CAR therapy can also be directed at a variety of tumor-associated antigens including, for example, 4-1BB, 5T4, adenocarcinoma antigen, alpha-fetoprotein, BAFF, B-lymphoma cell, C242 antigen, CA125, carbonic anhydrase 9 (CA-IX), C-MET, CCR4, CD152, CD19, CD20, CD21, CD22, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, alpha 5β1integrin, integrin αvβ3, MORAb-009, MS4A1, MUC1, mucin CanAg, Nglycolylneuraminic acid, NPC-1C, PDGF-Rα, PDL192, phosphatidylserine, prostatic carcinoma cells, RANKL, RON, ROR1, SCH 900105, SDC1, SLAMF7, TAG-72, tenascin C, TGF β2, TGF-β, TRAIL-R1, TRAIL-R2, tumor antigen CTAA16.88, VEGFA, VEGFR-1, VEGFR2, 707-AP, ART-4, B7H4, BAGE, β-catenin/m, Bcr-abl, MN/C IX antibody, CAMEL, CAP-1, CASP-8, CD25, CDC27/m, CDK4/m, CT, Cyp-B, DAM, ErbB3, ELF2M, EMMPRIN, ETV6-AML1, G250, GAGE, GnT-V, Gp100, HAGE, HLA-A*0201-R170I, HPV-E7, HSP70-2M, HST-2, hTERT (or hTRT), iCE, IL-2R, ILS, KIAA0205, LAGE, LDLR/FUT, MAGE, MART-1/melan-A, MART-2/Ski, MC1R, myosin/m, MUM-1, MUM-2, MUM-3, NA88-A, PAP, proteinase-3, p190 minor bcr-abl, Pml/RARα, PRAME, PSA, PSM, PSMA, RAGE, RU1 or RU2, SAGE, SART-1 or SART-3, survivin, TPI/m, TRP-1, TRP-2, TRP-2/INT2, WT1, NY-Eso-1 or NY-Eso-B or vimentin.

Cancer vaccines include, for example, human papilloma virus (HPV) vaccine, dendritic cell vaccines (e.g., Provenge® for prostate cancer), tumor cell vaccines, antigen vaccines, oncolytic virus vaccines (e.g., Imlygic™), Non-Hodgkin's lymphoma and mantle cell lymphoma vaccine (e.g., BioVaxID™), breast cancer vaccine (e.g., Neuvax™), brain cancer vaccine (e.g., DCVax™, CDX-110™), pancreatic cancer vaccine (e.g., GVAX Pancreas, HyperAcute™ Pancreas), colorectal cancer vaccine (e.g., Imprime PGG®), bladder cancer vaccine (e.g., BCG™), solid tumor vaccine (e.g., OK432™), lung cancer and gastrointestinal cancer vaccine (e.g., PSK™), cervical cancer vaccine (e.g., Schizophyllan™), and stomach cancer vaccine (e.g., Lentinan™).

Other immunotherapies for treating cancer include, for example, an IL-2 diphtheria toxin fusion protein (e.g., Ontak®),

### Types of Cancer

Cancers that can be treated with the methods described herein, include, for example, the approved indications for the FDA approved immunotherapies, such as melanoma, non-small cell lung cancer, Head and Neck squamous cell cancer, classical Hodgkin's lymphoma, primary mediastinal large B-cell lymphoma, urothelial carcinoma, microsatellite instability-high cancer, gastric cancer, cervical cancer, hepatocellular carcinoma, Merkel Cell carcinoma, renal cell carcinoma (Keytruda®); advanced or metastatic urothelial carcinoma, unresectable, stage III non-small cell lung cancer (Imfinzi®); unresectable or metastatic melanoma, metastatic non-small cell lung cancer, advanced renal cell carcinoma, classical Hodgkin's lymphoma, recurrent or metastatic squamous cell carcinoma, advanced or metastatic urothelial carcinoma, microsatellite instability high, or mismatch repair deficient metastatic colorectal cancer, hepatocellular carcinoma (Opdivo®); urothelial carcinoma, non-small cell lung cancer, triple negative breast cancer, small cell lung cancer (Tecentriq®); metastatic Merkel cell carcinoma (Bavencio®); unresectable of metastatic melanoma, advanced renal cell carcinoma, microsatellite instability high, or mismatch repair deficient metastatic colorectal cancer (Yervoy®); refractory diffuse B-cell lymphoma, relapsed or refractory acute lymphoblastic leukemia (Kymriah®); or diffuse large B-cell lymphoma, primary mediastinal B-cell lymphoma, High grade B-cell lymphoma (Yescarta®).

Cancers that can be treated with the methods described herein, also include, for example the indications under development such as, acute myeloid leukemia, bladder cancer, squamous cell carcinoma of the head and neck, chronic lymphocytic leukemia, multiple myeloma, metastatic solid malignancies (Lirilumab™); or melanoma, advanced colorectal cancer, advanced Chordoma, metastatic melanoma, gastro/esophageal cancer, solid tumors, gastric cancer, advanced renal cell cancer, advanced non-small cell lung cancer (Relatlimab™).

Other cancers that can be treated with the methods herein include, for example, sarcoma, carcinoma, melanoma, chordoma, malignant histiocytoma, mesothelioma, glioblastoma, neuroblastoma, medulloblastoma, malignant meningioma, malignant schwannoma, leukemia, lymphoma, myeloma, myelodysplastic syndrome, myeloproliferative disease. In some embodiments, the cancer is a leukemia, lymphoma, myeloma, myelodysplastic syndrome, and/or myeloproliferative disease.

### Vaccines

Vaccines can be substances used to stimulate a protective immune response in a subject (e.g., an antibody response or a cell mediated response) and provide immunity against one or several diseases. Vaccines protect against more than many debilitating or life-threatening diseases/infectious agents, including for example, adenovirus, anthrax, cervical cancer, chicken pox, cholera, dengue, diphtheria, Haemophilus influenza, hepatitis A, hepatitis B, hepatitis E, HPV, influenza, Japanese encephalitis, malaria, measles, meningitis, meningococcal (MenACWY), serogroup B meningococcal, mumps, pneumococcus, polio, rabies, rotavirus, rubella, shingles, small pox, tetanus, tuberculosis, typhoid, varicella, whooping cough, and yellow fever.

Vaccines can be prepared from the causative agent of a disease, its products, or a synthetic substitute, treated to act as an antigen without inducing the disease. Examples of vaccine types include, for example, live or attenuated vaccines (e.g., measles, mumps, rubella, varicella, influenza, rotavirus, zoster, yellow fever), inactivated or killed vaccines (e.g., polio, hepatitis A, rabies), toxoid (inactivated toxoid) vaccines (e.g., diphtheria, and tetanus), and subunit/conjugate vaccines (e.g., hepatitis B, influenza, Haemophilus influenza type b, pertussis, pneumococcal, meningococcal, HPV).

Attenuated vaccines can be made in several different ways. Some of the most common methods involve passing the disease-causing virus through a series of cell cultures or animal embryos (typically chick embryos). Using chick embryos as an example, the virus is grown in different embryos in a series. With each passage, the virus becomes better at replicating in chick cells, but loses its ability to replicate in human cells. A virus targeted for use in a vaccine may be grown through—“passaged” through—upwards of 200 different embryos or cell cultures. Eventually, the attenuated virus will be unable to replicate well (or at all) in human cells, and can be used in a vaccine. All of the methods that involve passing a virus through a non-human host produce a version of the virus that can still be recognized by the human immune system, but cannot replicate well in a human host. When the resulting vaccine virus is given to a human, it will be unable to replicate enough to cause illness, but will still provoke an immune response that can protect against future infection.

Killed or inactivated vaccines can be created by inactivating a pathogen, typically using heat or chemicals such as formaldehyde or formalin. This destroys the pathogen's ability to replicate, but keeps it “intact” so that the immune system can still recognize it. (“Inactivated” is generally used rather than “killed” to refer to viral vaccines of this type, as viruses are generally not considered to be alive.) Because killed or inactivated pathogens can't replicate at all, they can't revert to a more virulent form capable of causing disease (as discussed above with live, attenuated vaccines). However, these vaccines tend to provide a shorter length of protection than live vaccines, and are more likely to require boosters to create long-term immunity.

Immunizations created using inactivated toxins are called toxoids. Toxoids can actually be considered killed or inactivated vaccines, but are sometimes given their own category to highlight the fact that they contain an inactivated toxin, and not an inactivated form of bacteria.

Both subunit and conjugate vaccines contain only pieces of the pathogens they protect against. Subunit vaccines use only part of a target pathogen to provoke a response from the immune system. This may be done by isolating a specific protein from a pathogen and presenting it as an antigen on its own. The acellular pertussis vaccine and influenza vaccine (in shot form) are examples of subunit vaccines. Another type of subunit vaccine can be created via genetic engineering. Conjugate vaccines can be made using a combination of two different components. Conjugate vaccines, however, are made using pieces from the coats of bacteria. These coats are chemically linked to a carrier protein, and the combination is used as a vaccine.

### Pathogens

Pathogenic organisms are capable of causing disease in a subject. A human pathogen is capable of causing illness in humans. Common examples of pathogenic organisms include specific strains of bacteria such as, for example, Actinomyces israelii, Bacillus anthracis, Bacteroides fragilis, Bordetella pertussis, Borrelia, Brucella, Campylobacter jejuni, Chlamydophila psittaci, Corynebacterium diphtheria, Ehrlichia, Enterococcus, Francisella tularensis, Haemophilus influenza, Helicobacter pylori, Klebsiella pneumoniae, Legionella pneumophila, Leptospira species, Listeria monocytogenes, Mycobacterium, Mycoplasma pneumoniae, Pseudomonas aeruginosa, Nocardia asteroids, Rickettsia rickettsia, Salmonella, Shigella, Treponema pallidum, Vibrio cholera, Yersinia pestis, Listeria E. coli, Staphylococcus, Streptococcus, Neisseria, Clostridia, Chlamydia, mycoplasmas.

Pathogenic organisms also include viruses such as, for example, adenoviruses, herpesviruses, influenza, hepatitis, poxviruses, papovaviruses, paramyxoviruses, coronaviruses, picornaviruses, Reoviruses, togaviruses, flaviviruses, arenaviruses, rhabdoviruses, retroviruses, hepadnaviruses, Cryptosporidium.

Pathogenic organisms include fungi such as, for example, Candida, Aspergillus, Cryptococcus, Histoplasma, Pneumocystis, and Stachybotrys.

Pathogens also include the above organisms which are the target of vaccines.

Anti-pathogen therapies can include, for example, antibiotics for bacterial pathogens, anti-viral therapies for viral pathogens, and anti-fungal therapies for fungal pathogens. Antibodies can also be administered for the treatment of certain infectious diseases caused by bacteria, viruses or fungi.

Treatments Using iAge

Subjects with cancer who are candidates for immunotherapy (as described above) have their blood drawn and an iAge and CRS are calculated as described above. If the subject's iAge places them in the youngest iAge quartile for their age group (see Table 1) they can be classified as responders and move forward with the immunotherapy. If the subject's iAge places them in the middle two quartiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be treated with the immunotherapy. Subjects whose Jak-STAT activity places them in the lower three quartiles can be classified as nonresponders and are treated to lower iAge (and increase their Jak-STAT score) into a responder group. If the subject's iAge places them in the oldest quartile, they can be classified as nonresponders and are treated to lower their iAge (see below) into a responder group of a younger iAge quartile.

Alternatively, if the subject's iAge places them in the youngest iAge quintile for their age group (see Table 1) they can be classified as responders and move forward with the immunotherapy. If the subject's iAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be treated with the immunotherapy. Subjects whose Jak-STAT activity places them in the lower three quartiles can be classified as nonresponders and are treated to lower iAge (and increase their Jak-STAT score) into a responder group. If the subject's iAge places them in the oldest quintile, they can be classified as nonresponders and are treated to lower their iAge (see below) into a responder group of a younger iAge quintile.

Still alternatively, if the subject's iAge places them in the youngest iAge tertile for their age group (see Table 1) they can be classified as responders and move forward with the immunotherapy. If the subject's iAge places them in the middle tertile, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be treated with the immunotherapy. Subjects whose Jak-STAT activity places them in the lower three quartiles can be classified as nonresponders and are treated to lower iAge (and increase their Jak-STAT score) into a responder group. If the subject's iAge places them in the oldest tertile, they can be classified as nonresponders and are treated to lower their iAge (see below) into a responder group of a younger iAge tertile.

Subjects who are candidates for vaccinations (e.g., the elderly) can have their blood drawn and an iAge and CRS are calculated as described above. If the subject's iAge places them in the youngest iAge quartile for their age group (see Table 1) they can be classified as responders and move forward with the vaccination. If the subject's iAge places them in the middle two quartiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be vaccinated. Subjects whose Jak-STAT activity places them in the lower three quartiles can be classified as nonresponders and are treated to lower iAge (and increase their Jak-STAT score) into a responder group. If the subject's iAge places them in the oldest quartile, they can be classified as nonresponders and are treated to lower their iAge (see below) into a responder group of a younger iAge quartile. Subjects classified as nonresponders can also be treated with higher doses of vaccines and/or more aggressive vaccine formulations (e.g., cocktails of antigens, adjuvants, and/or immunostimulants) to account for the immunosenescence in the subject.

Alternatively, if the subject's iAge places them in the youngest iAge quintile for their age group (see Table 1) they can be classified as responders and move forward with the vaccination. If the subject's iAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be vaccinated. Subjects whose Jak-STAT activity places them in the lower three quartiles can be classified as nonresponders and are treated to lower iAge (and increase their Jak-STAT score) into a responder group. If the subject's iAge places them in the oldest quintile, they can be classified as nonresponders and are treated to lower their iAge (see below) into a responder group of a younger iAge quintile. Subjects classified as nonresponders can also be treated with higher doses of vaccines and/or more aggressive vaccine formulations (e.g., cocktails of antigens, adjuvants, and/or immunostimulants) to account for the immunosenescence in the subject.

Still alternatively, if the subject's iAge places them in the youngest iAge tertile for their age group (see Table 1) they can be classified as responders and move forward with the vaccination. If the subject's iAge places them in the middle tertile, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can be vaccinated. Subjects whose Jak-STAT activity places them in the lower three quartiles can be classified as nonresponders and are treated to lower iAge (and increase their Jak-STAT score) into a responder group. If the subject's iAge places them in the oldest tertile, they can be classified as nonresponders and are treated to lower their iAge (see below) into a responder group of a younger iAge tertile. Subjects classified as nonresponders can also be treated with higher doses of vaccines and/or more aggressive vaccine formulations (e.g., cocktails of antigens, adjuvants, and/or immunostimulants) to account for the immunosenescence in the subject.

Subjects who have been exposed to a pathogenic organism, are infected with a pathogenic organism, and/or are susceptible to infection by a pathogenic organism can have their blood drawn and an iAge and CRS are calculated as described above. If the subject's iAge places them in the youngest iAge quartile for their age group (see Table 1) they can be classified as responders and move forward with standard treatment for the pathogenic organism. If the subject's iAge places them in the middle two quartiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can move forward with standard treatment for the pathogenic organism. Subjects whose Jak-STAT activity places them in the lower three quartiles can be classified as nonresponders and are treated to lower iAge (and increase their Jak-STAT score) into a responder group. If the subject's iAge places them in the oldest quartile, they can be classified as nonresponders and are treated to lower their iAge (see below) into a responder group of a younger iAge quartile. Subjects classified as nonresponders can also be treated with more aggressive therapies and/or higher doses of therapeutics to account for the immunosenescence in the subject.

Alternatively, if the subject's iAge places them in the youngest iAge quintile for their age group (see Table 1) they can be classified as responders and move forward with standard treatment for the pathogenic organism. If the subject's iAge places them in the middle three quintiles, the subject's blood cells (e.g., CD4+ and CD8+ cells) are stimulated and Jak-STAT activity is measured (see, e.g., Example 1 below). Subject's whose Jak-STAT activity places them in the highest quartile can be classified as responders and can move forward with standard treatment for the pathogenic organism. Subjects whose Jak-STAT activity places them in the lower three quartiles can be classified as nonresponders and are treated to lower iAge (and increase their Jak-STAT score) into a responder group. If the subject's iAge places them in the oldest quintile, they can be classified as nonresponders and are treated to lower their iAge (see below) into a responder group of a younger iAge quintile. Subjects classified as nonresponders can also be treated with more aggressive therapies and/or higher doses of therapeutics to account for the immunosenescence in the subject.

Still alternatively, if the subject's iAge places them in the youngest iAge tertile for their age group (see Table 1) they can be classified as responders and move forward with standard treatment for the pathogenic organism. If the subject's iAge places them in the middle tertile, the subject's blood cells (e.g., CD4+ and CD8+ cells) are stimulated and Jak-STAT activity is measured (see, e.g., Example 1 below). Subject's whose JakSTAT activity places them in the highest quartile can be classified as responders and can move forward with standard treatment for the pathogenic organism. Subjects whose JakSTAT activity places them in the lower three quartiles can be classified as nonresponders and are treated to lower iAge (and increase their Jak-STAT score) into a responder group. If the subject's iAge places them in the oldest tertile, they can be classified as nonresponders and are treated to lower their iAge (see below) into a responder group of a younger iAge tertile. Subjects classified as nonresponders can also be treated with more aggressive therapies and/or higher doses of therapeutics to account for the immunosenescence in the subject.

Agents for Lowering iAge

In addition to using iAge to classify patients (FIG. 3), it can be used to derive individual inflammatory profiles by comparing subject's individual protein levels with those of a population (e.g., of similar chronological age). The resulting signatures (or barcodes) are used for protein-compound association (PCI) analysis using the drugbank database (www.drugbank.ca) and a personalized initial therapy to reduce iAge can be generated (FIG. 3). Patients following personalized recommendations can be monitored weekly for changes in iAge until they reach optimal levels (below group average for a given age bracket) and they convert into a responder to treatment phenotype (FIG. 3). The patient is then classified as a responder and is suitable for immunotherapy treatment.

A subject may reduce their iAge with treatments that lower the levels of TRAIL, IFNG, GROA, IL2, TGFA, PAIL and/or LIF to their optimal levels for a person's chronological age. A subject may also reduce their iAge with treatments that raise the levels of MIG, EOTAXIN, LEPTIN, IL-1B, or MIP1A to their optimal levels for a person's age.

A subject may also reduce their iAge by reducing any systemic chronic inflammation, using any of the following, whether alone or in combination: (1) pharmacological treatment, including without limitation anti-inflammatory drugs (NSAIDs such as, for example, aspirin, ibuprofen, naproxen, diclofenac, celecoxib, oxaprozin, piroxicam, indomethacin, meloxicam, fenoprofen, diflunisal, etodolac, ketorolac, meclofenamate, nabumetone) or corticosteroids (e.g., glucocorticoids, mineralocorticoids); (2) neutraceuticals or nutritional supplements, including without limitation fish oil, lipoic acid, and curcumin, or spices/herbs such as ginger, garlic, turmeric, hyssop, cannabis, Harpagophytum procumbens, and cayenne; (3) dietary change, including without limitation increasing the intake of foods that are high in antioxidants and polyphenols, such as olive oil, leafy greens (e.g., kale and spinach), broccoli, avocados, green tea, bell peppers, chili peppers, mushrooms, dark chocolate, cocoa, tomatoes, fatty fish (e.g., salmon, sardines, herring, anchovies, and mackerel), nuts (walnuts and almonds), and fruits (e.g., cherries, blackberries, blueberries, raspberries, strawberries, grapes, and oranges), and/or decreasing the intake of foods that can increase inflammation such as refined carbohydrates (e.g., white bread and pastries), high-fructose corn syrup, refined sugar, processed and packaged food, fried foods, red meat, excessive alcohol, and processed meat; and (4) lifestyle changes including without limitation eliminating or reducing smoking and alcohol intake, maintaining a healthy body weight, and reducing stress levels.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: iAge Correlates with Naïve CD8(+) T Cells and with the Ex Vivo Jak-STAT Signaling Responses to Stimulation

The frequency of circulating naïve CD8(+) T cells decline with high iAge (A) and iAge can predict poor ex vivo Jak-STAT signaling responses to stimulation (B and C). A total of 96 cytokine-cell-STAT combinations were analyzed with respect of a subject's iAge. These included eight cell types: B cells, CD4(+) T cells (and their CD45(+) and (−) subsets), CD8(+) T cells (and their CD45(+) and (−) subsets), and monocytes; four cytokines: Interleukin-6 (IL-6), IL-10, IL-21 and Interferon-alpha; and three STAT proteins (STAT1, 3 and 5). FIG. 1B: volcano plot, result of a multiple regression analysis with permutation tests to estimate false discovery rates (Benjamini-Hochberg FDR) (y-axis) as a function of the regression coefficients obtained for iAge after adjusting for Age, Gender and cytomegalovirus status. FIG. 1C: normalized ex vivo CD8(+) T cell phosphor-STAT-1 responses to Interleukin-6. The lower tertile for iAge shows significantly more robust responses than the higher tertile for iAge (C).

iAge is negatively correlated with naïve CD8(+) T cells and with the ex vivo JakSTAT signaling responses to stimulation.

### Example 2: Stratification of Cancer Patients Using iAge and CRS

A blood sample is obtained from patients prior to immunotherapy treatment. Serum and immune cells are separated by standard methods. Serum samples are used to measure protein concentration for inflammatory age (iAge) determination; and cells are cytokine-stimulated ex vivo to measure phosphorylation of intracellular signal transducer and activator of transcription (STAT) proteins to derive a cytokine response score (CRS). iAge and CRS can independently predict patient's response to immunotherapy treatment. FIG. 2 shows a flow diagram of this process.

iAge and CRS can be used to stratify cancer patients prior to treatment as responders versus non-responder for immunotherapy.

### Example 3: Stratification of Cancer Patients Using iAge

iAge can be used to classify cancer patients into responder and non-responders to immunotherapy treatment (A), and to derive iAge individual inflammatory protein signature (barcode), which is fed to iAge personalized recommendation engine to create an individualized initial therapy aimed to reduce iAge, inform medical decision and hence, convert those non-responder patients into responder patients (suitable for immunotherapy) (B). FIG. 3 shows a flow diagram of this process.

iAge is used to stratify patients for cancer immunotherapy and help convert nonresponders into responder for immunotherapy.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A method of treating a patient with an infectious disease, comprising the steps of: selecting a patient with an iAge in the youngest tertile for a chronological age of the patient; and administering an effective amount of an anti-pathogen therapy to the patient.

16. The method of claim 15, wherein the iAge is in the youngest quartile.

17. The method of claim 15, wherein the iAge is in the youngest quintile.

18. The method of claim 15, wherein the anti-pathogen therapy is an antibiotic, an anti-viral, or an antibody.

19. A method vaccinating a patient, comprising the steps of: selecting a patient with an iAge in the youngest tertile for a chronological age of a group of patients to receive a vaccine; and administering an effective amount of the vaccine to the patient.

20. The method of claim 19, wherein the iAge is in the youngest quartile.

21. The method of claim 19, wherein the iAge is in the youngest quintile.

22. The method of claim 19, wherein the vaccine is an influenza vaccine, a hepatitis vaccine, or an anti-malarial vaccine.

23. The method of claim 15, wherein the patient has a bacterial pathogen.

24. The method of claim 23, wherein the bacterial pathogen is a Bacillus anthracis, a Bordetella pertussis, a Legionella pneumophila, a Listeria monocytogenes, a Mycoplasma pneumoniae, a Pseudomonas aeruginosa, a Salmonella, a Shigella, a Vibrio cholera, a Staphylococcus, a Streptococcus, a Neisseria, a Clostridia, a Chlamydia, or a mycoplasma.

25. The method of claim 23, wherein the anti-pathogen therapy is an antibiotic.

26. The method of claim 23, further comprising the step of administering an agent to the patient that lowers the iAge of the patient.

27. The method of claim 15, wherein the patient has a virus.

28. The method of claim 27, wherein the virus is a herpesviruses, an influenza, a hepatitis, a poxvirus, a coronavirus, or a retrovirus.

29. The method of claim 27, wherein the anti-pathogen therapy is a antiviral.

30. The method of claim 27, further comprising the step of administering an agent to the patient that lowers the iAge of the patient.

31. The method of claim 19, wherein the vaccine is a live virus vaccine, an attenuated virus vaccine, an inactivated vaccine, a subunit vaccine or a conjugate vaccine.

32. The method of claim 31, wherein the vaccine protects from a measles, a mumps, a rubella, a varicella, an influenza, a rotavirus, a zoster, or a yellow fever.

33. The method of claim 31, wherein the inactivated vaccine is for a polio, a hepatitis A, or a rabies.

34. The method of claim 31, wherein the conjugate vaccine is for a hepatitis B, an influenza, a Haemophilus influenza type b, a pertussis, a pneumococcal, a meningococcal, or a HPV.
